(19) United States
(12) Patent Application Publication
(10) Pub. No.: US 2022/0307949 A1
(43) Pub. Date: Sep. 29, 2022

(54) CAPTURING DEVICE

(71) Applicant: DAIKIN INDUSTRIES, LTD., (US)

(72) Inventors: Yuta SASAI, Osaka-shi, Osaka (JP); Toshio TANAKA, Osaka-shi, Osaka (JP); Kiyoshi KUROI, Osaka-shi, Osaka (JP); Atsuo NOZAKI, Koriyama-shi, Fukushima (JP)

(21) Appl. No.: 17/840,248

(22) Filed: Jun. 14, 2022

Related U.S. Application Data
(63) Continuation of application No. PCT/JP2020/046942, filed on Dec. 16, 2020.

(30) Foreign Application Priority Data
Dec. 16, 2019 (JP) 2019-226170

(51) Int. Cl. G01N 1/22

(52) U.S. Cl. CPC G01N 1/2273; G01N 1/2205

(57) ABSTRACT

A collection apparatus collects a collection target in air in a target space. The collection apparatus includes an air passage having an inlet and an outlet, a carrier disposed in the air passage, and a plurality of collectors disposed in the air passage. The carrier carries the air. The plurality of carriers collects the collection target in the air carried by the carrier. The plurality of collectors collects different types of collection targets.

### CROSS-REFERENCE TO RELATED APPLICATIONS

This is a continuation of International Application No. PCT/JP2020/046942 filed on Dec. 16, 2020, which claims priority to Japanese Patent Application No. 2019-226170, filed on Dec. 16, 2019. The entire disclosures of these applications are incorporated by reference herein.

### BACKGROUND

### Technical Field

The present disclosure relates to a collection apparatus.

### Background Art

It has been known that substances suspended in indoor space, such as microorganisms, allergens, and gas components, cause sick house syndrome and allergic symptoms. A collection apparatus that collects the microorganisms and other substances suspended in the air in the indoor space has been developed to identify the substances causing such symptoms. Japanese Unexamined Patent Publication No. 2012-26954 discloses a collection apparatus having a collection surface to collect formaldehyde and volatile organic compounds in the air.

### SUMMARY

A first aspect of the present disclosure is directed to a collection apparatus that is configured to collect a collection target in air in a target space. The collection apparatus includes an air passage having an inlet and an outlet, a carrier disposed in the air passage, and a plurality of collectors disposed in the air passage. The carrier is configured to carry the air. The plurality of carriers is configured to collect the collection target in the air carried by the carrier. The plurality of collectors is configured to collect different types of collection targets.

### BRIEF DESCRIPTION OF THE DRAWINGS

FIG. 1 is a perspective view illustrating how a collection apparatus according to an embodiment is placed in an indoor space.

FIG. 2 is a schematic cross-sectional view illustrating the configuration of the collection apparatus.

FIG. 3 is a cross-sectional view taken along line III-III in FIG. 2.

FIG. 4 is a diagram illustrating relationship between a controller and various devices.

FIG. 5 is a block diagram illustrating the configuration of the controller.

FIG. 6 is a graph illustrating the operation of the collection apparatus.

FIG. 7 is an enlarged side view illustrating part of the configuration of a collection apparatus according to a first variation.

FIG. 8 is a diagram illustrating relationship between a controller and various devices according to the first variation.

FIG. 9 is a flowchart of collection time control using a dust sensor.

FIG. 10 is a longitudinal cross-sectional view illustrating the configuration of a collection apparatus according to a second variation.

FIG. 11 is a view corresponding to a cross-sectional view taken along line X-X in FIG. 3, illustrating a collection apparatus according to another embodiment.

FIG. 12A, FIG. 12B and FIG. 12C are vertical cross-sectional views illustrating the configuration of the collection apparatus and a storage container. FIG. 12A shows the collection apparatus not stored in the storage container. FIG. 12B shows the storage container.

FIG. 12C shows the collection apparatus stored in the storage container.

### DETAILED DESCRIPTION OF EMBODIMENT(S)

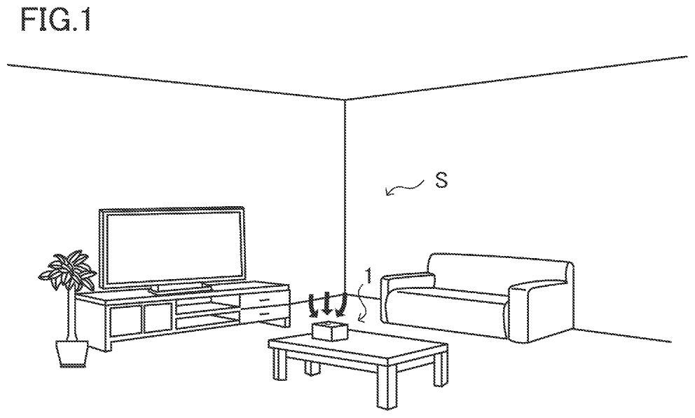

Embodiment(s) of the present invention will be described with reference to the drawings. The following description of embodiment(s) is merely beneficial examples in nature, and is not intended to limit the scope, applications, or use of the present disclosure. Note that the terms in the following description which indicate directions, such as “upper,” “top,” “lower,” “bottom,” “right,” “left,” “front.” and “rear” refer to the directions shown in the drawings unless otherwise specified. Arrows shown in FIGS. 1 to 3, 7, and 10 indicate an example of a flow direction of the air.

As illustrated in FIG. 1, a collection apparatus (1) of the present disclosure is placed in an indoor space (S) which is a target space of general housing, such as houses and apartments. The collection apparatus (1) collects collection targets in the air in the indoor space (S). The collection targets include solid components and gas components. The collected collection targets are subjected to subsequent analysis. The analysis includes, for example, qualitative analysis and quantitative analysis. The qualitative analysis is to examine the types of the collection targets contained in the air in the indoor space (S). The quantitative analysis is to examine the concentrations of various types of collection targets contained in the air in the indoor space (S).

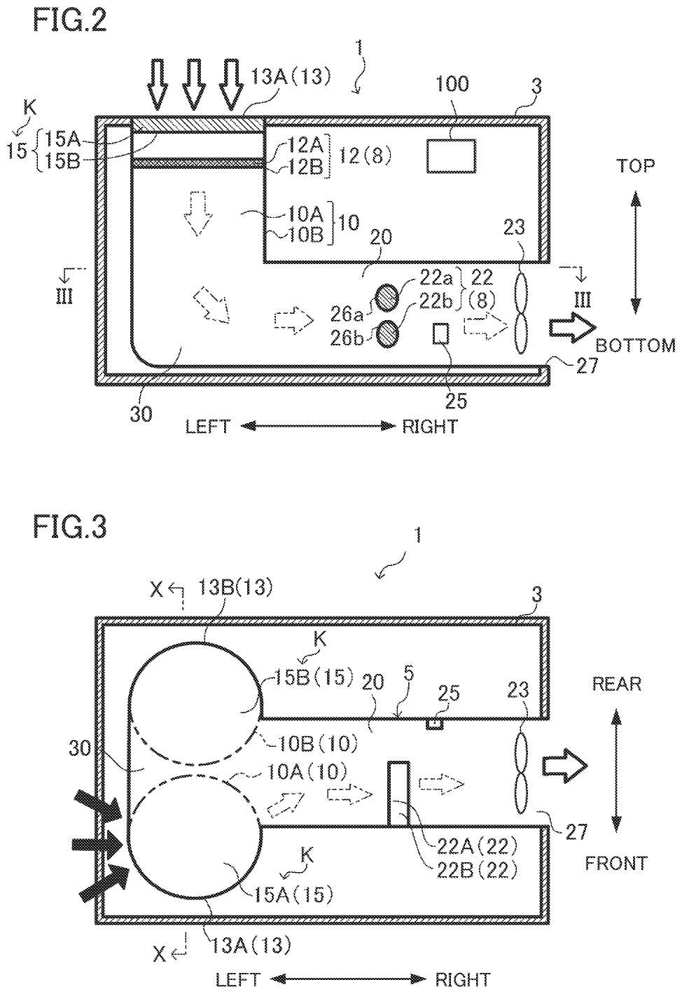

As illustrated in FIGS. 2 and 3, the collection apparatus (1) includes a casing (3), a first air passage (5), a first fan (23), a sampler (8), a shutter (15), an air speed sensor (25), and a controller (100).

### Casing

The casing (3) is hollow. The casing (3) is formed in a rectangular parallelepiped shape. The casing has two first inlets (13) and a single first outlet (27). The two inlets (13) are formed on a top surface of the casing (3). The two first inlets (13) are formed closer to the left side of the casing (3). The two first inlets (13) are aligned in a front-rear direction. The two first inlets (13) includes a front first inlet (13A) and a rear first inlet (13B). The front first inlet (13A) is formed closer to the front side of the casing (3). The rear first inlet (13B) is formed closer to the rear side of the casing (3). The first outlet (27) is formed on a right side surface of the casing (3).

### First Air Passage

The first air passage (5) is formed in the casing (3). The first air passage (5) is formed to extend from the two first inlets (13) to the first outlet (27). Specifically, the first air passage (5) has two first channels (10), a second channel (20), and an intermediate channel (30).

The two first channels (10) include a front first channel (10A) and a rear first channel (10B). The front first channel (10A) extends downward from the front first inlet (13A). The rear first channel (10B) extends downward from the rear first inlet (13B). Lower ends of the two first channels (10) are at the center of the height of the casing (3).

The second channel (20) extends leftward from the first outlet (27). A left end of the second channel (20) is located leftward of the center of the casing (3).

The intermediate channel (30) is a channel that allows downstream ends of the two first channels (10) to communicate with an upstream end of the second channel (20). Specifically, one end of the intermediate channel (30) is branched into two, which are connected to the lower ends of the two first channels (10). The other end of the intermediate channel (30) is connected to the left end of the second channel (20).

A first mounting port (26A) and a second mounting port (26B) are formed on an inner surface of the second channel (20). The first mounting port (26A) and the second mounting port (26B) are holes to which a first gas sampler (22A) and a second gas sampler (22B), which will be described later, are mounted. The first mounting port (26A) and the second mounting port (26B) are disposed one above the other.

### First Fan

The first fan (23) is a carrier that carries the air in the indoor space (S) to the first air passage (5). The first fan (23) is disposed downstream of the sampler (8) in the second channel (20) near the first outlet (27).

### Sampler

The sampler (8) is a collector that collects the collection targets contained in the air. The sampler (8) is disposed in the first air passage (5). Time for which the air passes through the sampler (8) and the amount of air that passes through the sampler (8) vary depending on the type of the collection target to be analyzed. The time for which the air passes and the amount of passing air are determined under a predetermined rule. The predetermined rule may be an official method of analysis, for example. The sampler (8) includes a gas collector (22) and a solid collector (12).

The gas collector (22) collects gas components in the air in the indoor space (S). The gas collector (22) includes a plurality of gas collectors. Specifically, the gas collector (22) includes a first gas sampler (22A) and a second gas sampler (22B). The first gas sampler (22A) and the second gas sampler (22B) are disposed upstream of the first fan (23) in the second channel (20). The first gas sampler (22A) and the second gas sampler (22B) are disposed not to partially or entirely overlap with each other when viewed in a flow direction of the air in the second channel (20). Specifically, the first gas sampler (22A) and the second gas sampler (22B) are disposed one above the other. More specifically, the first gas sampler (22A) and the second gas sampler (22B) are disposed side by side in a direction orthogonal to the flow direction of the air in the second channel (20).

The first gas sampler (22A) includes an adsorber having a high capability of adsorbing formaldehyde in the air. In other words, the first gas sampler (22A) shows a higher ability to collect formaldehyde than to collect other gas components different from formaldehyde.

The second gas sampler (22B) includes an adsorber having a high capability of adsorbing ammonia in the air. In other words, the second gas sampler (22B) shows a higher ability to adsorb ammonia than to adsorb other gas components different from ammonia.

The solid collector (12) collects solid components suspended in the air in the indoor space (S). The solid collector (12) has a higher ability to collect the solid components than the gas collector (22). The solid collector (12) includes a first solid sampler (12A) and a second solid sampler (12B).

The first solid sampler (12A) is disposed upstream of the gas collector (22). Specifically, the first solid sampler (12A) is disposed near the first inlet (13) in the front first channel (10A). The first solid sampler (12A) has a filter for collecting the solid components. The filter of the first solid sampler (12A) has a high ability to collect mold in the air flowing through the front first channel (10A). In other words, the first solid sampler (12A) shows a higher ability to collect the mold than to collect other solid components different from the mold.

The second solid sampler (12B) is disposed upstream of the gas collector (22). Specifically, the second solid sampler (12B) is disposed near the first inlet (13) in the rear first channel (10B). The second solid sampler (12B) has a filter for collecting the solid components. The filter of the second solid sampler (12B) has a high ability to collect mold in the air flowing through the rear first channel (10B). In other words, the second solid sampler (12B) shows a higher ability to collect the mites than to collect other solid components different from the mites.

### Shutter

The shutter (15) is a first mechanism (K) that switches a target first channel (10) between the two first channels (10) and allow the air to enter the target first channel (10) and flow through the second channel (20). Specifically, the shutter (15) is a first opening/closing mechanism provided upstream of each of the first solid sampler (12A) and the second solid sampler (12B) in the two first channels (10). More specifically, the shutter (15) includes a first shutter (15A) and a second shutter (15B). The first shutter (15A) is disposed at the first inlet (13) in the front first channel (10A). The second shutter (15B) is disposed at the first inlet (13) in the rear first channel (10B).

The front first channel (10A) corresponding to the first shutter (15A) or the rear first channel (10B) corresponding to the second shutter (15B) is opened and closed. For example, when the target first channel (10) is the front first channel (10A), the first shutter (15A) is opened and the second shutter (15B) is closed. When the target first channel (10) is the rear first channel (10B), the first shutter (15A) is closed and the second shutter (15B) is opened.

### Air Speed Sensor

The air speed sensor (25) is disposed upstream of the gas collector (22) in the second channel (20). The air speed sensor (25) detects the speed of the air flowing through the first air passage (5) when the first fan (23) is operated.

### Controller

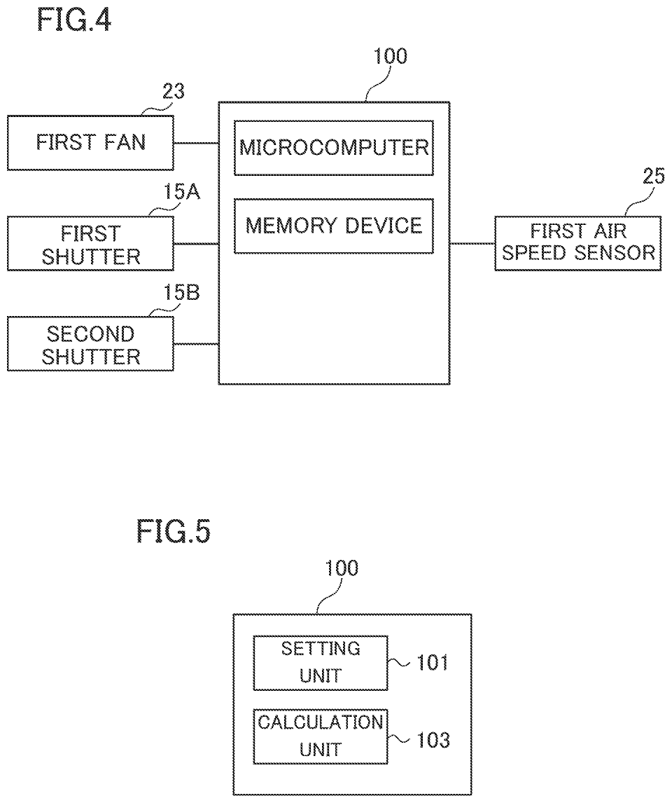

As illustrated in FIG. 4, the controller (100) includes a microcomputer mounted on a control board and a memory device (specifically, a semiconductor memory) that stores software for operating the microcomputer.

The controller (100) transmits and receives signals to and from the air speed sensor (25), the first shutter (15A), the second shutter (15B), and the first fan (23). These devices and the controller are connected to each other in a wireless or wired manner.

As illustrated in FIG. 5, the controller (100) includes a setting unit (101) and a calculation unit (103).

The setting unit (101) sets the amount (M) of air passing through the gas collector (22) and the solid collector (12), or travel time (ΔT) for which the air passes through the gas collector (22) and the solid collector (12). The mold present in the air is analyzed based on a predetermined amount of air (M) passing through the first solid sampler (12A). The setting unit (101) sets the predetermined amount (M) of air passing through the first solid sampler (12A) as a first air amount (M1). The mites present in the air are analyzed based on a predetermined travel time (ΔT) for which the air passes through the second solid sampler (12B). The setting unit (101) sets the predetermined travel time (ΔT) for which the air passes through the second solid sampler (12B) as second time (ΔT2). The gas components present in the air, such as formaldehyde and ammonia, are analyzed based on a predetermined travel time (ΔT) for which the air passes through the first gas sampler (22A) and the second gas sampler (22B). The setting unit (101) sets a predetermined travel time (ΔT) for which the air passes through the first gas sampler (22A) and the second gas sampler (22B) as third time (ΔT3).

The calculation unit (103) calculates a target air speed in the first air passage (5). The target air speed is determined based on the first air amount (M1), the second time (ΔT2), and the third time (ΔT3) which are set by the setting unit (101). Specifically, the calculation unit (103) calculates the first time (ΔT1), which is the collection time of the first solid sampler (12A), from the difference between the second time (ΔT2) and the third time (ΔT3). The calculation unit (103) calculates the target air speed from the first time (ΔT1) and the first air amount (M1).

### Operation

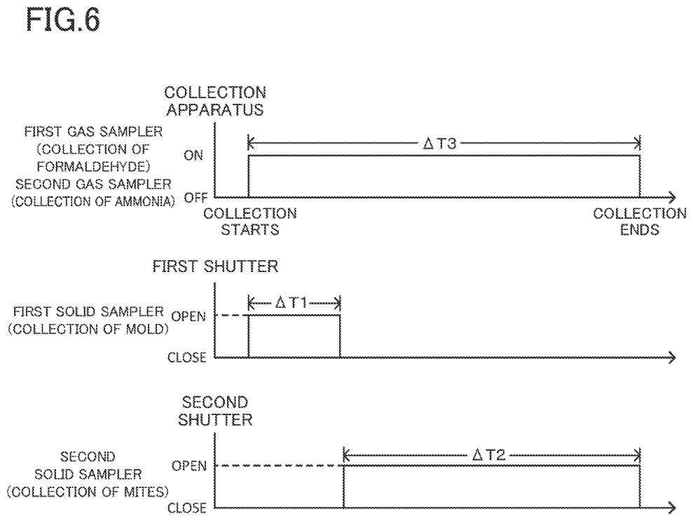

An example of the operation of the collection apparatus (1) will be specifically described with reference to FIG. 6.

The controller (100) sets various values. Specifically, the setting unit (101) sets the first air amount (M1) to 5 liters, the second time (ΔT2) to 175 minutes, and the third time (ΔT3) to 180 minutes. The calculation unit (103) calculates that the first time (ΔT2) is five minutes from the difference between the second time (ΔT3) and the third time (ΔT1). The calculation unit (103) calculates that the target air speed is one liter/min from the first time (ΔT1) and the first air amount (M1).

When the collection apparatus (1) is operated, the controller (100) opens the first shutter (15A) and closes the second shutter (15B). The controller (100) controls the number of rotations of the first fan (23) so that the air flows through the first air passage (5) at the target air speed. In this state, the first solid sampler (12A) collects the mold in the air sucked from the front first inlet (13A), but the second solid sampler (12B) collects no collection target in the air. The first gas sampler (22A) collects formaldehyde in the air that has passed through the first solid sampler (12A). The second gas sampler (22B) collects ammonia in the air that has passed through the first solid sampler (12A).

When five minutes which is the first time (ΔT1) has elapsed, the controller (100) closes the first shutter (15A) and opens the second shutter (15B). In this state, the first solid sampler (12A) ends the collection, and the second solid sampler (12B) starts collecting the mites in the air sucked from the first inlet (13). The first gas sampler (22A) collects formaldehyde in the air that has passed through the second solid sampler (12B). The second gas sampler (22B) collects ammonia in the air that has passed through the second solid sampler (12B).

When 175 minutes which is the second time (ΔT2) has elapsed, the controller (100) closes the first shutter (15A) and the second shutter (15B). The controller (100) ends the operation of the collection apparatus (1).

### Advantages of Embodiment

The collection apparatus (1) of the above embodiment collects the collection targets in the air in the target space (S). The collection apparatus (1) includes the air passage (5) having the inlet (13) and the outlet (27), the carrier (23) disposed in the air passage (5) and configured to carry the air, and the plurality of collectors (8) disposed in the air passage (5) and configured to collect the collection targets in the air carried by the carrier (23). The plurality of collectors (8) are configured to collect different types of collection targets.

The air in the indoor space (S), which is the target space, contains different types of collection targets, such as microorganisms, allergens, and gas components that cause sick house syndrome and allergic symptoms. For the examination of the substances that cause the sick house syndrome and the allergic symptoms, the collection targets in the air in the indoor space (S) need to be collected efficiently. For this purpose, a collection apparatus customized for various types of collection targets can be used. However, collection of different types of collection targets requires the collection apparatus for each collection target. This complicates the collection process because the different apparatuses need to be operated. A space for placing the multiple apparatuses is also required. It also takes time because these apparatuses need to be sent to an analysis institute. The collection apparatus (1) of the present embodiment includes a plurality of collectors (8) that collect different types of collection targets. Specifically, each collector (8) has a high ability to collect a specific collection target. Thus, the collection apparatus can efficiently collect different types of collection targets.

Each of the collectors (8) is disposed in the single air passage (5). Thus, the different types of collection targets can be collected at the same time in a short time.

Further, there is no need to place multiple collection apparatuses corresponding to the different types of collection targets. This makes the required space small, and saves time and labor for operating the apparatuses.

The single collection apparatus can collect different types of collection targets. This can reduce the parts count of the collection apparatus, and can lower the manufacturing cost. The collection apparatus can be downsized.

Just the single collection apparatus (1) is sent to the institute that analyzes the collection targets. For example, a workload for sending the apparatus can be reduced as compared to the case where multiple collection apparatuses customized for different types of collection targets are used and sent to the institute.

In the embodiment, each of the plurality of collectors (8) has the solid collector (12) configured to collect the solid component as the collection target and the gas collector (22) configured to collect the gas component as the collection target.

This configuration allows the collection apparatus (1) to collect the collection targets in the air in the target space (S) separated into the solid components and the gas components. This eliminates the need to separate the solid components and the gas components. Thus, the solid components and the gas components are quickly submitted for analysis.

Each collector (8) can collect the gas components or the solid components in a specific manner. Thus, the collection apparatus can efficiently collect different types of collection targets.

In the embodiment, the solid collector (12) is disposed upstream of the gas collector (22) in the first air passage (5).

In this configuration, the solid components as the collection targets are collected first, and then the gas components as the collection targets are collected. This can keep the solid components from adhering to the gas collector (22). As a result, the gas collector (22) can collect the gas components with increased collection efficiency. Similarly, the solid collector (12) can collect the solid components with increased collection efficiency.

In the embodiment, the solid collector (12) is configured to collect the suspended microorganisms and the suspended allergens as the solid components.

This configuration allows the collection apparatus to collect the mold which is one of the suspended microorganisms and the mites which is one of the suspended allergens. Thus, the types of mold and mites suspended in the air in the indoor space (S) can be identified.

In the embodiment, the gas collector (22) is configured to collect volatile organic compounds and odor gases as the gas components.

This configuration allows the collection apparatus to collect formaldehyde which is one of the volatile organic compounds and ammonia as one of the odor gases.

In the embodiment, the first air passage (5) includes the two first channels (10) and the single second channel (20). The downstream ends of the two first channels (10) communicate with the upstream end of the second channel (20). The solid collector (12) is disposed in each of the two first channels (10), and the gas collector (22) is disposed in the second channel (20).

In this configuration, the second channel (20) can be a downstream passage communicating with the two first channels (10). Specifically, the two first channels (10) merge on the downstream side and communicate with the second channel (20). Thus, the first solid sampler (12A) and the gas collector (22) collect the collection targets in the air flowing into the front first channel (10A). The second solid sampler (12B) and the gas collector (22) collect the collection targets in the air flowing into the rear first channel (10B). This allows the gas collector (22) to collect the collection targets contained in the air that has passed through the first solid sampler (12A) and the collection targets contained in the air that has passed through the second solid sampler (12B). As a result, the parts count of the collection apparatus can be reduced as compared with, for example, a collection apparatus including two air passages and the solid collector and the gas collector disposed in each of the air passages.

In the embodiment, the collection apparatus further includes the first mechanism (K) configured to switch the target first channel (10) between the two first channels (10) and allow the air to enter the target first channel (10) and flow through the second channel (20).

In this configuration, switching of the target first channel (10) allows continuous collection of different types of solid components. For example, the first mechanism (K) allows the target front first channel (10A) to communicate with the second channel (20). At this time, the rear first channel (10B) does not communicate with the second channel (20). Thus, the first solid sampler (12A) and the gas collector (22) collect the collection targets in the air. Thereafter, the first mechanism (K) switches the target first channel (10). Specifically, the target rear first channel (10B) communicates with the second channel (20). At this time, the front first channel (10A) does not communicate with the second channel (20). Thus, the second solid sampler (12B) and the gas collector (22) collect the collection targets in the air. Switching the target first channel (10) by the first mechanism (K) in this way allows continuous collection of two different types of solid components.

Since the second channel (20) communicates with the two first channels (10), the gas collector (22) can continuously collect the gas components from the start of collection by one of the solid collectors (12) to the end of collection by the other solid collector (12).

In the embodiment, the first mechanism (K) includes the first opening/closing mechanism (15) provided upstream of each of the solid collectors (12) in the plurality of first channels (10) and configured to open and close the first channel (10) corresponding to the first opening/closing mechanism (15).

In this configuration, opening and closing the first opening/closing mechanism (15) allows the switching of the target first channel (10). Specifically, the first mechanism (K) includes the shutter (15) serving as the first opening/closing mechanism (15). The shutter (15) includes the first shutter (15A) and the second shutter (15B). The first shutter (15A) is disposed upstream of the first solid sampler (10A) in the front first channel (12A). The second shutter (15B) is disposed upstream of the second solid sampler (10B) in the rear first channel (12B). When the first shutter (15A) is opened and the second shutter (15B) is closed, the air can flow only into the front first channel (10A) corresponding to the first shutter (15A). When the second shutter (15B) is opened and the first shutter (15A) is closed, the air can flow only into the rear first channel (10B) corresponding to the second shutter (15B). Thus, switching the first shutter (15A) and the second shutter (15B) between the open state and the closed state allows the switching of the target first channel (10).

The shutter (15) is disposed upstream of the solid collector (12). Thus, when the shutter (15) is closed, the air is kept from entering the first inlet (13). The shutter (15) is opened only for a set predetermined period, and the solid collector (12) does not collect the collection targets beyond the predetermined period. This can improve the accuracy of analysis of various types of solid components present in the air in the indoor space (S).

In the embodiment, the inlet (13) opens upward.

This configuration allows the collection apparatus to suck the air above. Thus, the solid components falling down from above can be efficiently sucked.

In the embodiment, the outlet (27) opens laterally.

The collection apparatus (1) of this configuration having the outlet (27) disposed on the side surface requires no space for blowing the air out below the collection apparatus (1). If the outlet (27) is disposed on a lower surface of the collection apparatus (1), for example, legs or any other parts need to be attached to the lower surface to provide a space for blowing the air out. Thus, the collection apparatus (1) requires no legs or any other parts, and can be made compact.

### First Variation

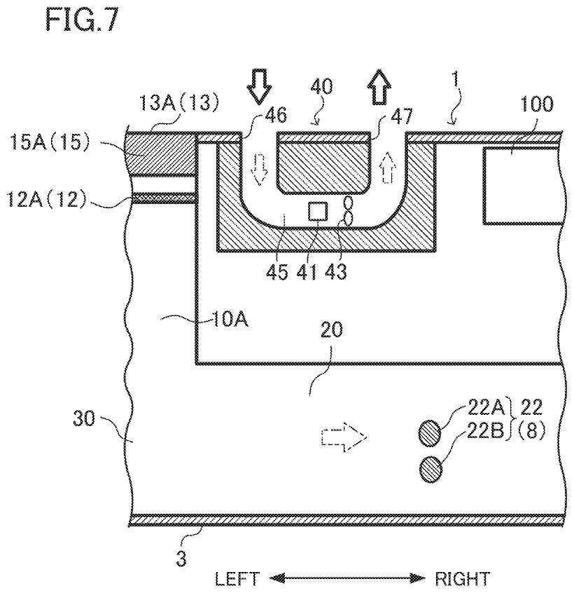

As illustrated in FIG. 7, a collection apparatus (1) of a first variation includes a dust detector (40) that detects the concentration of dust in the air in the indoor space (S). The controller (100) determines time for the first solid sampler (12A) to collect the mold based on the dust concentration detected by the dust detector (40). For example, the collection time when the dust concentration is within a predetermined concentration range is set as a first collection time. When the dust concentration is below the predetermined concentration range, it is determined that the amount of mold contained in the air in the indoor space (S) is equal to or less than the detection limit. In this case, the controller (100) sets the time for mold collection to be shorter than the first collection time. When the dust concentration exceeds the predetermined concentration range, it is determined that the mold contained in the air in the indoor space (S) exceeds the upper limit value of detection. Also in this case, the controller (100) sets the time for mold collection to be shorter than the first collection time. Differences from the collection apparatus (1) of the embodiment will be described in detail below.

### Dust Detector

The dust detector (40) is provided in an upper portion of the casing (3). The dust detector (40) includes a second air passage (45), a second fan (43), and a dust sensor (41).

A second inlet (46) and a second outlet (47) are provided near the center of a top surface of the casing (3). The second air passage (45) extends from the second inlet (46) to the second outlet (47).

The second fan (43) is disposed in the second air passage (45). The second fan (43) carries the air in the indoor space (S) to the second air passage (45).

The dust sensor (41) detects the concentration of dust in the air flowing through the second air passage (45).

### Controller

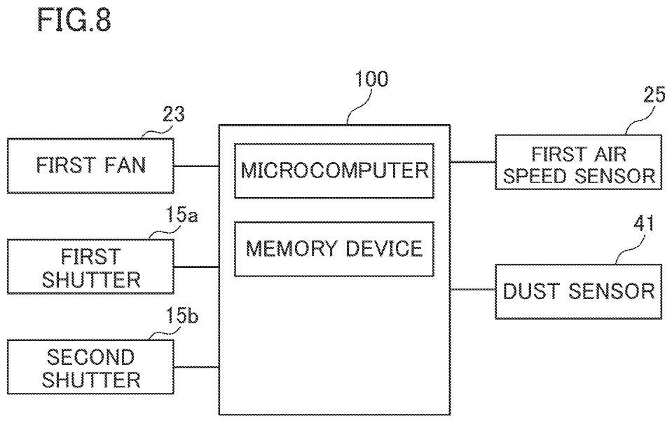

As illustrated in FIG. 8, the controller (100) is connected to the dust sensor (41) and various other devices constituting the collection apparatus (1) via communication lines. The controller (100) sets the collection time for the first solid sampler (12A) based on the dust concentration in the indoor space (S) detected by the dust sensor (41). Specifically, the controller (100) stores a predetermined dust concentration range. The predetermined concentration range is an appropriate concentration range for measuring the concentration of mold present in the air in the indoor space (S). The predetermined concentration range is, for example, 10 μg/m³ to 100 μg/m³.

### Control of Collection Tune for First Solid Sampler Based on Dust Concentration

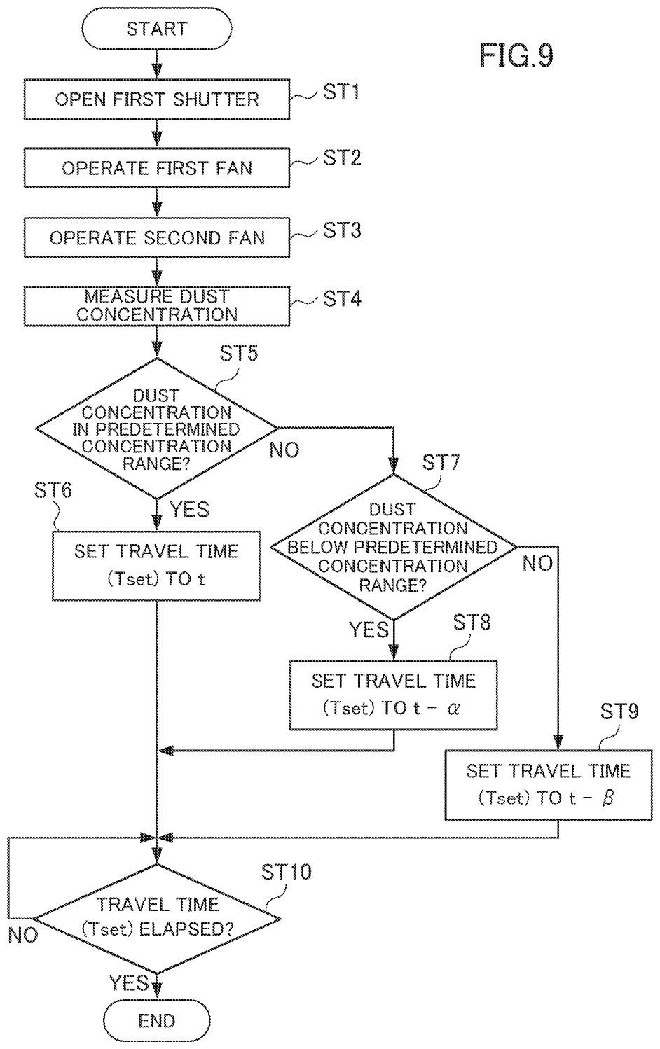

An example of control of the collection time for the first solid sampler (12A) based on the dust concentration will be described with reference to FIG. 9.

When the setting unit (101) sets the first air amount (M1) passing through the first solid sampler (12A), the operation of the collection apparatus (1) starts. The first air amount (M1) is a value inputted by a user's operation.

In Step ST1, the controller (100) opens the first shutter (15A).

In Step ST2, the controller (100) operates the first fan (23). The first solid sampler (12A) starts collecting the mold.

In Step ST3, the controller (100) operates the second fan (43).

In Step ST4, the dust sensor (40) starts measuring the dust concentration.

In Step ST5, the controller (100) determines whether the dust concentration is within a predetermined concentration range. If the answer is YES, the process proceeds to Step ST6, and the controller (100) sets a travel time (Tset) for the air to pass through the first solid sampler (12A). The travel time (Tset) in this step is t minutes, and t is five, for example. In the answer is NO, the process proceeds to Step ST7.

In Step ST7, the controller (100) determines whether the dust concentration is below the minimum value of the predetermined concentration range. The minimum value is 10 μg/m³. If the answer is YES, the process proceeds to Step ST8, and the controller (100) sets the travel time (Tset). The travel time (Tset) in this step is (t−α) minutes, and α represents a fixed value stored in advance in the setting unit (101). For example, if α is four, the transmit time (Tset) is one minute. If the answer is NO, the dust concentration is greater than the maximum value. The maximum value is 100 μg/m³. In this case, the process proceeds to ST9, and the controller (100) sets the travel time (Tset). The travel time (Tset) in this step is (t−β) minutes, and β represents a fixed value stored in advance in the setting unit (101). For example, if β is two, the travel time (Tset) is three minutes. Note that t, α, and β meet t>α>β.

In Step ST10, the controller determines whether the travel time (Tset) for the air to pass through the first solid sampler (12A) has elapsed. If the answer is YES, the operation of the collection apparatus (1) ends. If the answer is NO, the process returns to Step ST10, and it is determined again whether the travel time (Tset) has elapsed.

In this variation, the collection time for the first solid sampler (12A) is set based on the dust concentration in the air in the indoor space (S). For example, when the dust concentration is less than 10 μg/m³, which is the minimum value of the predetermined concentration range, it is determined that almost no mold is present in the indoor space (S). The number of mold fungi that the first solid sampler (12A) can collect is equal to or less than the detection limit. Thus, the travel time is set to t−α (one minute), which is shorter than t (five minutes). This can shorten the collection time, and can finish the sampling operation quickly.

When the dust concentration is higher than 100 μg/m³, which is the maximum value of the predetermined concentration, it is determined that a relatively large amount of mold is present in the indoor space (S). The number of mold fungi that the first solid sampler (12A) collects exceeds the upper limit of detection. Thus, the travel time is set to t−β (three minutes), which is shorter than t (five minutes). This can shorten the collection time, and can finish the sampling operation quickly. The upper limit of detection is, for example, 300 cfu.

### Second Variation

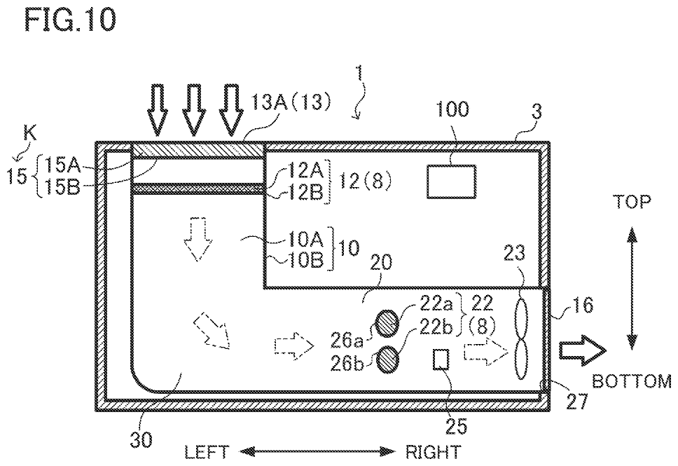

As illustrated in FIG. 10, a collection apparatus (1) of a second variation includes a third shutter (16). The third shutter (16) is a second opening/closing mechanism provided downstream of the first gas sampler (22A) and the second gas sampler (22B) in the second channel (20). The third shutter (16) opens and closes the second channel (20). Specifically, the third shutter (16) is disposed at the first outlet (27).

The third shutter (16) is connected to the controller (100) in a wireless or wired manner. The controller (100) controls the opening and closing of the third shutter (16). The controller (100) opens the third shutter (16) when the operation of the collection apparatus (1) starts. The controller (100) closes the third shutter (16) when the operation of the collection apparatus (1) ends.

Specifically, when the operation of the collection apparatus (1) starts, the controller (100) opens at least one of the first shutter (15A) or the second shutter (15B) and the third shutter (16). When the operation of the collection apparatus (1) ends, the controller (100) closes the first shutter (15A), the second shutter (15B), and the third shutter (16).

This can keep the gas components collected by the gas samplers (22A, 22B) from leaking outside from the first outlet (27) after the operation of the collection apparatus (1) ends. Thus, when the collection apparatus (1) is sent to a predetermined analysis institute to analyze the collection targets, the amount of collected gas components can be kept from decreasing due to the leakage of the gas components from the collection apparatus (1) during the transport of the collection apparatus (1). This can also keep the accuracy of measurement of the amount of collected gas components from decreasing.

### OTHER EMBODIMENTS

The foregoing embodiment may be modified as follows.

The solid components collected by the first solid sampler (12A) are not limited to the mold suspended in the air in the indoor space (S). The first solid sampler (12A) collects any suspended microorganisms in the air in the indoor space (S) as the solid components. Examples of the suspended microorganisms include bacteria and viruses.

The solid components collected by the second solid sampler (12B) are not limited to the mites suspended in the air in the indoor space (S). The second solid sampler (12B) may collect any suspended allergens in the air in the indoor space (S) as the solid components. Examples of the suspended allergens include pollen, dust with animal saliva, and hair.

The first solid sampler (12A) and the second solid sampler (12B) may collect the suspended microorganisms. The first solid sampler (12A) and the second solid sampler (12B) may collect the suspended allergens.

The solid components collected by the solid collector (12) are not limited to the suspended microorganisms and the suspended allergens, and may be minerals and organic compounds. Examples of the minerals include glass fibers. Examples of the organic compounds include diesel dust.

The solid collector (12) may include three or more solid samplers. This allows simultaneous collection of three or more types of solid components.

The gas components collected by the first gas sampler (22A) are not limited to formaldehyde in the air in the indoor space (S). The first gas sampler (22A) may collect volatile organic compounds in the air in the indoor space (S) as the gas components. The volatile organic compounds are so-called volatile organic compounds (VOC).

The gas components collected by the second gas sampler (22B) are not limited to ammonia in the air in the indoor space (S). The second gas samplers (22B) may collect odor gases as the gas components. Examples of the odor gases include hydrogen sulfide.

The first gas sampler (22A) and the second gas sampler (22B) may collect the volatile organic compounds. The first gas sampler (22A) and the second gas sampler (22B) may collect the odor gases.

It is only required that the first gas sampler (22A) and the second gas sampler (22B) be disposed not to partially or entirely overlap with each other when viewed in the flow direction of the air in the second channel (20). The first gas sampler (22A) and the second gas sampler (22B) may not be disposed side by side in the direction orthogonal to the flow direction of the air.

The gas collector (22) may include three or more gas samplers. This allows collection of three or more types of gas components.

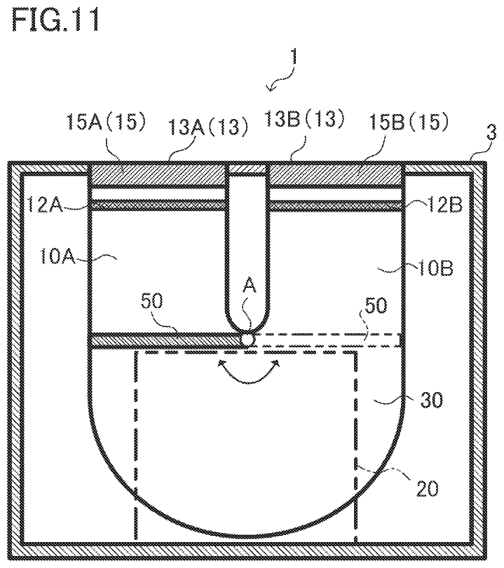

It is only required that the first mechanism (K) switches the target first channel (10) between the front first channel (10A) and the rear first channel (10B). For example, as illustrated in FIG. 11, the first mechanism (K) may be a damper (50). In this case, the damper (50) is disposed in the intermediate channel (30). The damper (50) moves about an axis A between a first position (solid line in FIG. 11) at the lower end of the front first channel (10A) and a second position (dotted line in FIG. 11) at the lower end of the rear first channel (10B). When the damper (50) is at the first position, the front first channel (10A) is closed, and the rear first channel (10B) and the second channel (20) communicate with each other. When the damper (50) is at the second position, the rear first channel (10B) is closed, and the front first channel (10A) and the rear first channel (10B) communicate with each other. Thus, moving the damper (50) between the first position and the second position can change the target first channel (10).

The collection apparatus (1) may include the first fan (23) in each of the front first channel (10A) and the rear first channel (10B). When each of the first fans (23) is operated, the first solid sampler (12A) and the second solid sampler (12B) can collect the targets independently. Thus, the control for closing one of the shutters (15A, 15B) while the other shutter (15A, 15B) is open is no longer necessary. As a result, both shutters (15A, 15B) can be opened, and the collection time can be shortened.

The collection apparatus (1) may have three or more first channels (10). This can increase the types of collection targets (solid components).

In the variations, when the dust concentration is below the minimum value, or above the maximum value, of the predetermined concentration range, it is only required that the collection time be shorter than the collection time when the dust concentration is within the predetermined concentration range. The travel time (Tset) may meet t>β≥α.

In the variations, the predetermined dust concentration range may be set by the user. Regarding the travel time (Tset) for the air to pass through the first solid sampler (12A), α and β may not be fixed values, and may be values that vary depending on the rotational speed of the first fan (23), or may be values inputted by the user.

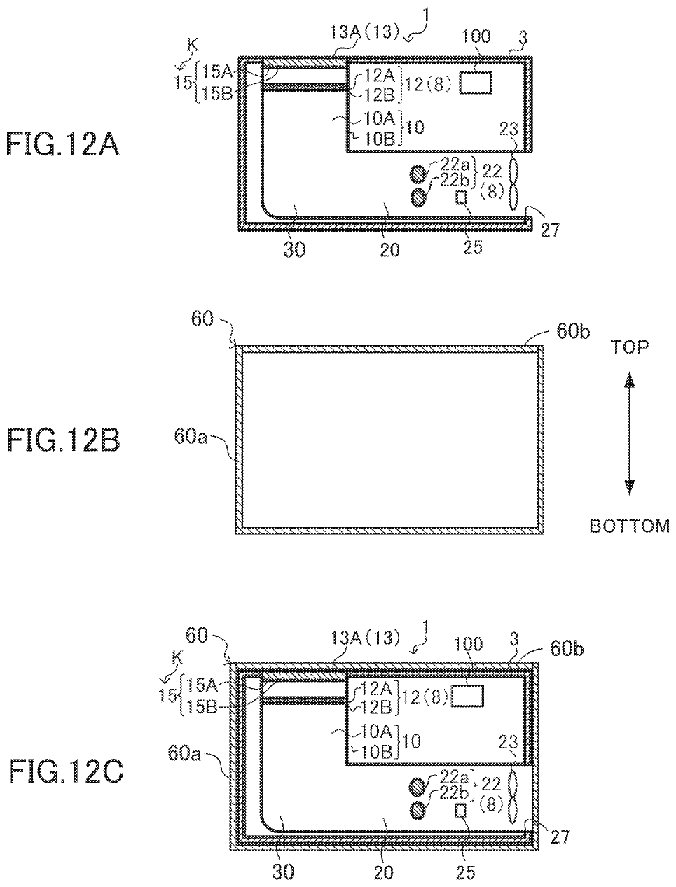

The collection apparatus (1) of the second variation may have no third shutter (16). In this case, as illustrated in FIG. 12C, the collection apparatus (1) (FIG. 12A) may be stored in a separate storage container (60) (FIG. 12B) after the operation of the collection apparatus (1) ends. Specifically, the storage container (60) is formed in a box shape. The storage container (60) has a storing portion (60a) having an open top and a lid (60a) covering the opening of the storing portion (60a). When the lid (60b) is attached to the storing portion (60a), the inside of the storage container (60) is sealed. As illustrated in FIG. 12C, when the collection apparatus (1) is stored in the storage container (60), the first outlet (27) is blocked by the sidewall of the storing portion (60a). The two first inlets (13) are blocked by the lid (60b). In this manner, when the collection apparatus (1) is stored in the storage container (60) after the operation ends, the gas components collected by the gas samplers (22A, 22B) can be kept from leaking outside from the first outlet (27).

The second opening/closing mechanism (16) may be a damper.

While the embodiments and variations thereof have been described above, it will be understood that various changes in form and details may be made without departing from the spirit and scope of the claims. The embodiments and the variations thereof may be combined and replaced with each other without deteriorating intended functions of the present disclosure. The ordinal numbers such as “first,” “second,” “third,” . . . , described above are used to distinguish the terms to which these expressions are given, and do not limit the number and order of the terms.

As can be seen in the foregoing, the present disclosure is useful for a collection apparatus.

1. A collection apparatus configured to collect a collection target in air in a target space, the collection apparatus comprising:
an air passage having an inlet and an outlet;
a carrier disposed in the air passage, the carrier being configured to carry the air; and
a plurality of collectors disposed in the air passage, the plurality of carriers being configured to collect the collection target in the air carried by the carrier,
the plurality of collectors being configured to collect different types of collection targets.

2. The collection apparatus of claim 1, wherein
the plurality of collectors include
a solid collector configured to collect a solid component as the collection target and
a gas collector configured to collect a gas component as the collection target.

3. The collection apparatus of claim 2, wherein
the solid collector is disposed upstream of the gas collector in the air passage.

4. The collection apparatus of claim 2, wherein
the solid collector is configured to collect at least one of a suspended microorganism, a suspended allergen, a mineral, and an organic compound as the solid component.

5. The collection apparatus of claim 2, wherein
the gas collector is configured to collect at least one of a volatile organic compound and an odor gas as the gas component.

6. The collection apparatus of claim 2, wherein
the air passage includes a plurality of first channels and a single second channel,
downstream ends of the plurality of first channels communicate with an upstream end of the second channel,
the solid collector is disposed in each of the plurality of first channels, and
the gas collector is disposed in the second channel.

7. The collection apparatus of claim 6, further comprising:
a first mechanism configured to switch a target first channel between the plurality of first channels and allow the air to enter the target first channel and flow through the second channel.

8. The collection apparatus of claim 7, wherein
the first mechanism includes a first opening and closing mechanism
provided upstream of each of the solid collectors in the plurality of first channels and
configured to open and close the first channel corresponding to the first opening and closing mechanism.

9. The collection apparatus of claim 1, wherein
the inlet opens upward.

10. The collection apparatus of claim 9, wherein
the outlet opens laterally.
